(19) United States
(12) Patent Application Publication
(10) Pub. No.: US 2005/0234501 A1
(43) Pub. Date: Oct. 20, 2005

(54) Braided intraluminal filter

(76) Inventor: David D. Barone, Lexington, MA (US)

Correspondence Address: MEDTRONIC VASCULAR, INC.;IP LEGAL DEPARTMENT, 3576 UNOCAL PLACE, SANTA ROSA, CA, 95403, US

(21) Appl. No.: 10/826,653

(22) Filed: Apr. 15, 2004

(51) Int. Cl. A61M 29/00

(52) U.S. Cl. 606/200

(57) ABSTRACT

A braided intraluminal filter having large inlet ports, and methods for making the filter. Selected braid filaments are severed such that they do not extend through a proximal section of the filter. The un-severed filaments are divided into groups that are intra-braided into slender strands forming the proximal section of the filter. Large inlet ports are defined between the slender strands.

### FIELD OF THE INVENTION

The present invention relates generally to intraluminal devices for capturing particulate in the vessel of a patient. More specifically, the invention relates to a temporary filter for capturing emboli in a blood vessel during an interventional vascular procedure and removing the captured emboli from the patient after completion of the procedure.

### BACKGROUND OF THE INVENTION

A variety of therapeutic modalities exists for treating vessel narrowings, which may comprise atherosclerotic plaque and/or thrombus, and which may be alternatively described as stenoses or lesions. Balloon angioplasty, stent deployment, atherectomy, and thrombectomy are well known techniques that have proven effective, alone or in combination, in the treatment of such stenoses. During each of these procedures, there is a risk that particulate dislodged by the procedure will migrate through the circulatory system to embolize in critical organs, causing ischaemic events such as infarction or stroke. Thus, practitioners have approached prevention of escaped emboli through use of occlusion devices, filters, lysing, and aspiration techniques. For example, it is known to remove embolic material by suction through an aspiration lumen in the treatment catheter or by capturing emboli in a filter or with an occlusion device.

Prior art intraluminal filters for protection against atheroembolization are associated with either a catheter or guidewire and are temporarily positioned downstream of the area to be treated. One prior art filter catheter includes a collapsible filter mounted distally of a dilatation balloon. Filter material is secured to resilient ribs, and a filter balloon is located between the catheter exterior and the ribs. Inflation of the filter balloon extends the ribs outward across the vessel to form a trap for fragments loosened by the dilatation balloon. When the filter balloon is deflated, the resilient ribs retract against the catheter to retain the fragments during withdrawal of the catheter.

Another prior art filter arrangement includes several filter elements fastened in a spaced-apart arrangement along the length of a catheter. This forms an open-mouthed, tubular, sock-like arrangement for capturing emboli. The filter is collapsed by spirally wrapping it around the flexible catheter.

Another prior art device has a filter made from a shape memory material. The device is deployed by moving the proximal end of the filter towards the distal end. It is collapsed and withdrawn by sliding a sheath over the filter and then removing the sheath and filter together.

Yet another prior art filter includes a filter mounted on the distal portion of a hollow guidewire or tube. A movable core wire is used to open and close the filter. The filter distal end is secured to the guide wire and the proximal end has an expandable rim formed by the core wire.

Other known collapsible intraluminal filters are formed from braided filaments. In some designs, the braided pattern has small interstices, making it unnecessary to cover the braid with a porous membrane or filter material. One example has a cylindrical central body for deployment against the wall of a vessel lumen. The downstream, or distal section of the braided filter is gathered or tapered for attachment to the shaft of a catheter or guidewire. At the upstream, or proximal section of the braided filter, individual struts extend between the cylindrical central body and the shaft. The proximal struts are affixed to the other elements of the assembly by joints such as welding, soldering or adhesive bonding.

In another known collapsible filter formed from braided filaments and having a cylindrical central body, both ends of the braided filter are gathered or tapered for attachment to the shaft of a catheter or guidewire. Holes substantially larger than the interstices are cut in a tapered end of the braided filter to form inlet ports. Alternatively, enlarged inlet ports may be formed by stretching selected interstices around shaping mandrels and heat-treating the filter to set the desired sizes and shapes of the ports.

Uncovered, uncut, braided filters are simple to manufacture, have a low collapsed profile, and provide good structural integrity because each filament extends continuously through the filter. However, a problem associated with known temporary filter arrangements is that all of the potentially embolic debris may not be trapped within the filter. Particulate can accumulate outside the filter on any upstream, or proximal surfaces between and/or around the inlet ports. As the filter is closed, debris accumulated outside the filter can escape past the filter and embolize. In a braided filter having uncut filaments, all the filaments must be bunched together between and around the inlet ports to occupy an unnecessarily large surface area on the upstream, or inlet, end of the filter. In such filter structures, the uninterrupted aggregation of braid filaments extending through the filter inlet end limits the sizes and shapes of inlet ports that can be formed by stretching interstices.

Therefore, there is a need for a temporary intraluminal filter having inlet ports that occupy as much area as possible on the upstream, or inlet, end of the filter to enhance entrapment of debris entrained with the bodily fluid flowing through the lumen. Stated another way, the filter surface between and/or around inlet ports should be minimized. Such a filter apparatus should also be simple to manufacture. Such a filter apparatus should also have good structural integrity.

There is also a need for an intraluminal catheter or guidewire having a temporary intraluminal filter with inlet ports that occupy as much area as possible on the upstream, or inlet, end of the filter to enhance entrapment of debris entrained with the bodily fluid flowing through the lumen. Other desirable features and characteristics of the present invention will become apparent from the detailed description below and the appended claims, taken in conjunction with the accompanying drawings.

### SUMMARY OF THE INVENTION

According to an aspect of the present invention, a braided intraluminal filter has large inlet ports. Selected braid filaments are severed such that they do not extend through a proximal section of the filter. The un-cut filaments are divided into groups that are intra-braided into slender strands forming the proximal section of the filter such that the large inlet ports are defined between the slender strands.

### BRIEF DESCRIPTION OF THE DRAWINGS

The appended drawings are illustrative of the particular embodiments of the invention and therefore do not limit its scope. They are presented to assist in providing a proper understanding of the invention. The present invention will hereinafter be described in conjunction with the drawings, wherein like reference numerals denote like elements. The drawings are not to scale

FIGS. 1A and 1B illustrate side views of a filter in accordance with the invention, shown in expanded and collapsed configurations, respectively;

FIGS. 2-4 are views showing the inlet ends of several embodiments of a filter in accordance with the invention;

FIG. 5 is an enlarged supplementary view of a portion of FIG. 1A;

FIG. 6 is a side view of a filter incorporating a retention member in accordance with the invention;

FIG. 7 is a partial side view of a filter guidewire assembly in accordance with the invention; and

FIGS. 8-11 illustrate various stages of a process for making a filter in accordance with the invention.

### DETAILED DESCRIPTION OF THE INVENTION

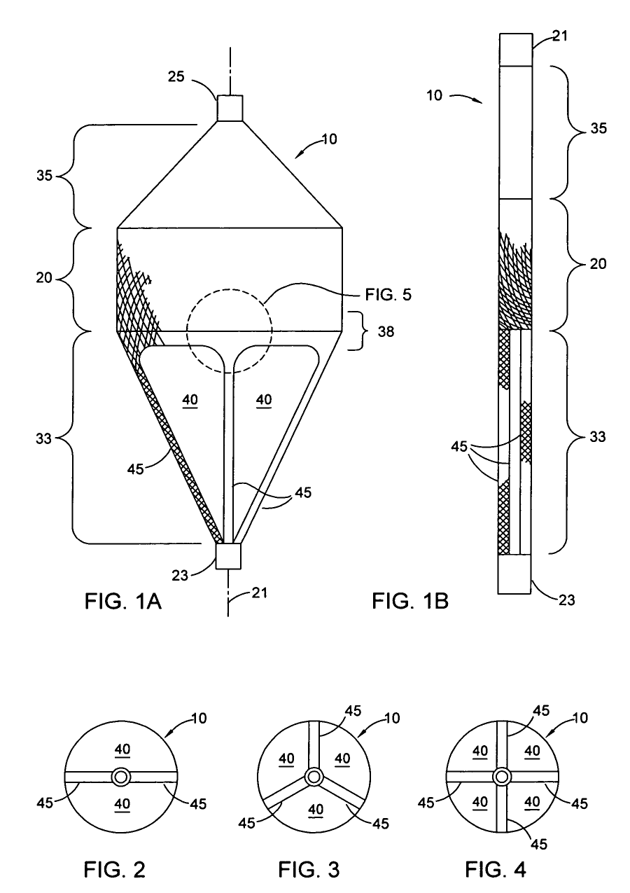

FIG. 1A shows filter 10 in an expanded configuration suitable for deployment of generally cylindrical filter body 20 into sealing apposition against the inner wall of a patient's vessel (not shown). The sealing contact is preferably maintained around the entire vessel wall to prevent any emboli from escaping past filter 10. Filter 10 includes longitudinal axis 21 extending through proximal and distal ends 23, 25, respectively. Flexible hollow filter 10 can be transformed into the collapsed configuration shown in FIG. 1B by drawing proximal and distal ends 23, 25 apart along axis 21. In the expanded configuration of FIG. 1A, distal section 35 tapers from the distal end of filter body 20 to filter distal end 25. Proximal section 33 tapers from junction region 38, where it adjoins the proximal end of filter body 20 to filter proximal end 23. Proximal section 33 comprises inlet ports 40 formed between strands 45.

Filter 10 comprises a generally tubular form made from a multiplicity of braided filaments. Filter body 20 and distal section 35 have a generally constant-pitch braid that is sufficiently dense so that no additional filter material is necessary for trapping potentially embolic particulates. In one example, 48 filaments can be densely braided to form interstices having a uniform pore size of approximately 75-125 microns. FIGS. 2, 3 and 4 depict views from the inlet ends of various inventive filter embodiments having two, three and four strands 45, respectively. The four-strand embodiment of FIG. 4 corresponds to the embodiment shown in the side view of FIG. 1A. The invention is not limited to the coaxially symmetry about longitudinal axis 21, as shown in the expanded configurations of FIGS. 1A and 2-4. Instead, axis 21 may be displaced off-center through filter body 20.

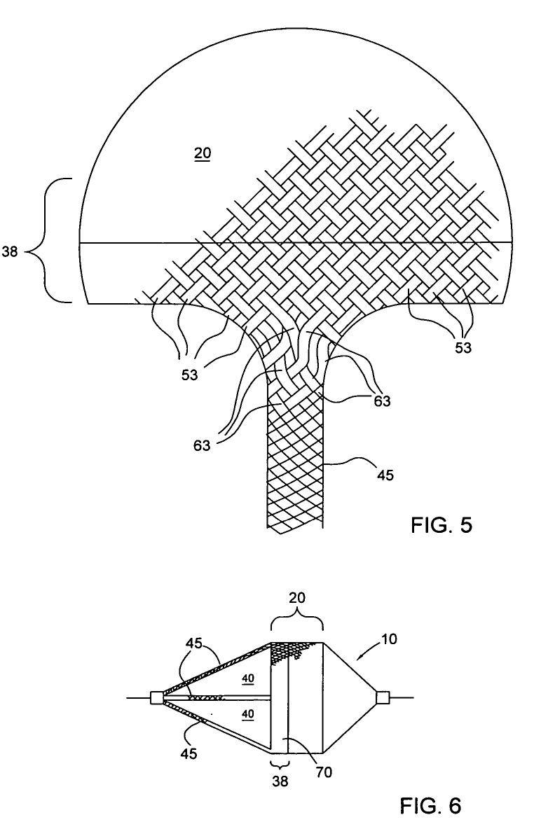

FIG. 5 illustrates, in enlarged view, a portion of junction 38 where one strand 45 joins filter body 20. Filter 10 comprises two collections, or arrays of braid filaments. A first array comprises filaments 53 that extend proximally from filter distal end 25 to junction region 38, where filaments 53 have been cut or severed such that they terminate and do not extend substantially into or through filter proximal section 33. A second array comprises filaments 63 that extend from filter distal end 25 to filter proximal end 23.

In filter distal end 25, distal section 35 and filter body 20, filaments 63 of the second array are inter-braided with filaments 53 of the first array. However, in filter proximal section 33, filaments 63 of the second array are divided into two or more groups, each of which is intra-braided to compose a strand 45. The intra-braiding of filaments 63 within each strand 45 is performed separately and subsequently from the original braiding of a tubular filter precursor, as will be described in further detail below.

By cutting away portions of filaments 53 so that they do not extend through filter proximal section 33, strands 45 comprising the remaining filaments 63 can be slender and have a small combined surface area. Thus, inlet ports 40, which are formed between strands 45, can be large in comparison to a braided filter having uncut filaments and inlet ports formed by stretched interstices, wherein all the filaments must be bunched together between and around the inlet ports.

FIG. 6 illustrates filter 10 further incorporating a retention member 70 for coupling the proximal terminus of the first array to the second array. Retention member 70 retains the cut ends of filaments 53 to prevent filter 10 from unraveling or to keep the cut ends from injuring the vessel wall. Retention member 70 may comprise a pattern of joints formed where filaments 53 overlap filaments 63. The joints between filaments 53, 63 may be formed by one or more joining methods such as adhesive bonding, heat bonding, melt bonding, soldering, brazing, welding, laser welding, resistance welding, spot welding or other methods suitable for joining the materials composing filaments 53, 63. Alternatively, retention member 70 may comprise an elastic encapsulating sleeve such as cast silicone or a thermoplastic elastomer (TPE) applied with a solvent carrier or by shrink-wrapping techniques. The encapsulating sleeve may have a short length along the circumference of junction region 38, or the encapsulating sleeve may extend distally from junction region 38 over all or part of generally cylindrical body 20.

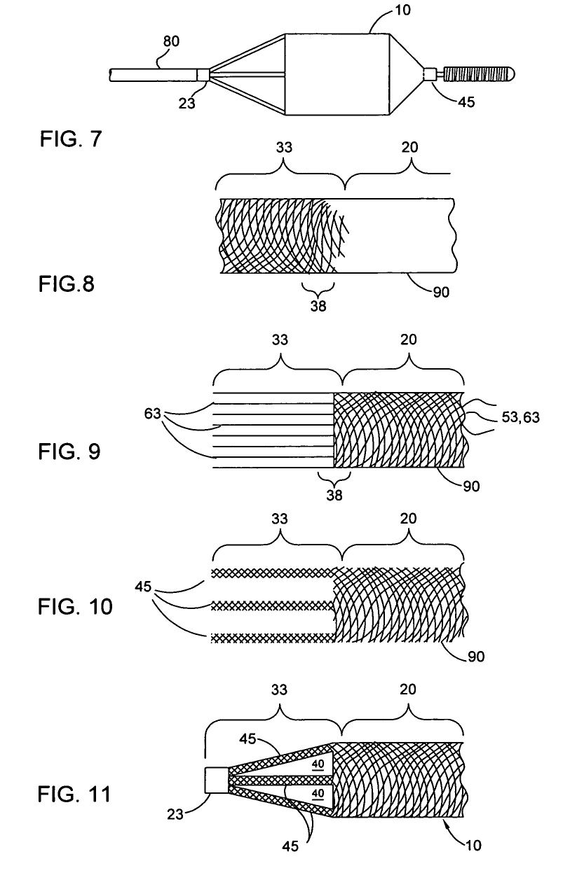

FIG. 7 shows filter 10 coupled about the distal end of catheter 80. Either filter proximal end 23, or distal end 25, or both ends may be fixedly, slidably or rotatably coupled to catheter 80, depending upon the desired mode of deployment and other characteristics. For example, catheter 80 may comprise a hollow wire-like shaft coupled to filter proximal end 23, and a movable core wire coupled to filter distal end 25. In this example, push-pull actuation of the shaft and core wire control relative movement of filter ends 23, 25, thus transforming filter 10 between expanded and collapsed configurations.

Filter 10 may be heat set to retain shape memory of the expanded configuration. Instead of using a push-pull actuation system, an outer sheath (not shown) may slide over catheter 80 and retain filter 10 in the collapsed configuration during delivery and retrieval. Both filter ends 23, 25 may also be rotatably coupled to catheter 80 such that catheter 80 can rotate freely within filter 10. In another example, both filter ends 23, 25 may be slidably coupled to catheter 80, which also has at least one stop element (not shown) to limit axial movement of filter 10 along catheter 80.

FIGS. 8-11 illustrate various stages of making filter 10. First, filter precursor tube 90 is braided from multiple filaments, as shown in FIG. 8. As will be understood by those of skill in the art, tubular braids generally comprise an equal number of filaments wound in the clockwise and counter-clockwise helical directions. The braid may have a “standard weave” wherein one filament crosses over and one filament crosses under, or a derivative “basket weave” wherein multiple filaments cross over and an equal number of filaments cross under. As shown in FIG. 8, regions that are intended to become filter proximal section 33, cylindrical body 20, and junction region 38 are demarcated along tube 90.

Next, a pattern for cutting of filaments 53 is devised, taking into consideration such design elements as the desired number of strands 45 in filter 10 and the number of filaments 63 required to provide at least a minimum structural strength in each strand 45. FIGS. 2, 3 and 4 illustrate various inventive filter embodiments having two, three and four strands 45, respectively. Increasing the number of strands 45 increases the lateral stability of filter 10 such that the filter inlet end can be reliably oriented transverse to the patient's vessel. However, increasing the number of strands 45 can decrease the size of each inlet port 40 until the largest embolic particles cannot enter filter 10. The cutting pattern will define how many, and which braided filaments in precursor tube 90 are to become severed filaments 53.

FIG. 9 illustrates another filter manufacturing stage, in which an exemplary cutting pattern entails cutting every other filament around the circumference of junction region 38 such that one-half of all braided filaments have become severed filaments 53. In another example, every third filament can be cut such that one-third of all filaments become severed filaments 53. In another cutting pattern, groups of adjacent filaments can be cut to become severed filaments 53, and other groups of adjacent filaments can remain uncut as continuous filaments 63, as shown in FIG. 5.

As shown in FIG. 9, the region of tube 90 that is intended to become filter proximal section 33 may be intentionally unraveled before or after filaments 53 are severed. Retention member 70, as shown in FIG. 6, can be incorporated into the filter assembly at any one of numerous manufacturing stages. Retention member 70 may be incorporated before filaments 53 are severed, to prevent the cut ends from fraying. Retention member 70 may also be incorporated to confine any intentionally unraveling to the region of tube 90 that is intended to become filter proximal section 33.

After filaments 53 have been cut, and the cut ends removed, then filaments 63 are gathered into groups. Each group of filaments 63 is intra-braided to form strands 45, as illustrated in FIG. 10. Intra-braiding, as the term is used herein, means the filaments of each group are braided exclusively within that group, and further that no strand 45 is braided with another strand 45. Thus, each strand 45 comprises a braided format of filaments 63 that is different from the braided format that formed precursor tube 90.

Expanding on the above example, every other one of 48 filaments is cut around the circumference of precursor tube 90. In the region intended to become proximal section 33, the remaining 24 filaments 63 are divided into four groups of six filaments 63 each. Each of the four groups of six filaments 63 is intra-braided to form a strand 45. Intra-braiding of strands 45 can be done manually or by a maypole-type braiding machine.

FIG. 11 shows strands 45 converging to form filter proximal end 23, which may comprise a ring or radiopaque band to assist in joining together strands 45. Alternatively, filter proximal end 23 may be formed by attaching strands 45 directly to a catheter shaft.

Filter 10 may have a mechanical memory to return to either the expanded, or the collapsed configuration. Such mechanical memory can be imparted to the filter material by thermal treatment to achieve a spring temper in stainless steel wire, for example, or to set a shape memory in a susceptible metal alloy such as a nickel-titanium (nitinol) alloy, or in a thermoplastic fiber. Heat treatments can be performed at the manufacturing final stage to set the overall shape of filter 10 and the slender profiles of strands 45. Alternatively, precursor tube 90 may also be heat set to prevent unraveling of the braid during subsequent manufacturing stages.

While at least one exemplary embodiment has been presented in the foregoing detailed description, it should be appreciated that numerous variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing the exemplary embodiment or exemplary embodiments. It should be understood that various changes can be made in the function and arrangement of elements without departing from the scope of the invention as set forth in the appended claims and the legal equivalents thereof.

1. A tubular intraluminal filter having proximal and distal ends, a longitudinal axis, a collapsed configuration and an expanded configuration, the expanded configuration having a generally cylindrical body and adjoining proximal and distal sections that taper from the cylindrical body to proximal and distal filter ends, respectively, wherein relative movement of the proximal and distal ends along the axis accompanies transformation of the filter between the collapsed configuration and the expanded configuration, the filter comprising:
a first array of braided filaments extending proximally from the filter distal end to a proximal terminus within a junction region between the cylindrical body and the tapered proximal section; and
a second array of filaments inter-braided with the first array and extending a full length of the filter, wherein the filter proximal section is defined by two or more strands, each strand being intra-braided from groups of filaments of the second array.

2. The filter according to claim 1 wherein the filter proximal section has two or more inlet ports defined by open spaces between the two or more strands

3. The filter according to claim 1 wherein filter pores are formed by interstices between the braided filaments.

4. The filter according to claim 1 wherein the proximal terminus of the first array is coupled to the second array by a retention member.

5. The filter according to claim 4 wherein the retention member comprises an elastic encapsulating sleeve.

6. The filter according to claim 4 wherein the retention member comprises joints formed where filaments of the first array overlap filaments of the second array.

7. The filter according to claim 6 wherein the joints are formed by one or more joining methods selected from adhesive bonding, heat bonding, melt bonding, soldering, brazing, welding, laser welding, resistance welding, and spot welding.

8. The filter according to claim 1 wherein the filter has a shape memory of the expanded configuration.

9. The filter according to claim 1 wherein the filter has a shape memory of the collapsed configuration.

10. A filter catheter comprising a flexible elongate shaft, a filter according to claim 1 mounted about a distal end of the shaft, and an actuation system for causing relative movement of the filter proximal and distal ends along the longitudinal axis of the filter.

11. A method of making an intraluminal filter, the method comprising:
braiding multiple filaments to form a filter precursor tube;
demarcating tube regions that are intended to become a proximal filter section, a cylindrical filter body, and a junction region there between;
severing selected filaments at the junction and removing severed filaments from the proximal filter section;
dividing the un-severed filaments in the proximal filter section into two or more groups;
intra-braiding each group of filaments into a corresponding strand; and
heat-treating the filter to set a selected shape thereof.

12. The method of making an intraluminal filter according to claim 11, further comprising coupling the ends of the severed filaments to adjacent filaments at the junction.

13. The method of making an intraluminal filter according to claim 11, further comprising converging the strands to form a proximal end of the filter.

14. The method of making an intraluminal filter according to claim 11, wherein the selected shape of the filter comprises a generally cylindrical body with proximal and distal ends tapering away from the body.
